(19) United States
(12) Patent Application Publication
(10) Pub. No.: US 2007/0105848 A1
(43) Pub. Date: May 10, 2007

(54) Methylene Blue Therapy of Parasitic Infections

(75) Inventors: Christopher Wood, Stoke Poges (GB); Nagy Habib, Heliopolis (GB)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Bioenvision, Inc.

(21) Appl. No.: 11/534,300

(22) Filed: Sep. 22, 2006

Related U.S. Application Data
(60) Provisional application No. 60/720,147, filed on Sep. 23, 2005.

(51) Int. Cl. A61K 31/5415; A61K 31/4706; A61K 31/513; A61K 31/185; A61K 31/29

(52) U.S. Cl. 514/224.5; 514/224.8; 514/305; 514/269; 514/503; 514/313; 514/553

(57) ABSTRACT

A method for using thiazine dyes, especially methylene blue, alone or in combination with low levels of light, to treat parasitic diseases is described. Examples of useful thiazine dyes are methylene blue, azure A, azure C, toluidine, and thionine. The preferred dye is methylene blue, administered orally twice a day. Since methylene blue absorbs in the red wavelengths, i.e., approximately 670 nm, which penetrates tissue much better than other lower wavelengths, light penetrating the skin to the capillaries at the surface can be used to enhance the activity of the dye. The thiazine dye can be provided in combination with other known antibiotics, anti-inflammatories, anti-parasitics, antifungals, and antivirals.

### CROSS-REFERENCE TO RELATED APPLICATIONS

This application claims priority to U.S. application Ser. No. 60/720,147, entitled “Methylene Blue Therapy of Parasitic Infections” by Christopher Wood and Nagy Habib filed Sep. 23, 2005.

### BACKGROUND OF THE INVENTION

This invention is generally in the area of methods for the treatment of parasitic diseases, and more specifically relates to the treatment of parasites using thiazine dyes, and in particular methylene blue.

Protozoa require the invasion of a suitable host to complete all or part of their life cycle. Such organisms are therefore termed parasites. Parasite infections affect millions of people world-wide afflicting considerable human suffering and economic hardship. Far from declining, many parasite infections are increasing throughout the world. The impact of Human Immunodeficiency Virus (HIV) and AIDS has seen the emergence of “new” opportunistic parasites as well as the increased prevalence of other recognized types. Climatic changes induced through global warming have aided the spread of many parasite diseases, whilst starvation and the breakdown in sanitation that accompanies war have seen the re-emergence of others. The appearance of drug resistance has also dramatically influenced the ability to treat and control many parasite diseases. In the United Kingdom parasite infections are relatively uncommon. However, outbreaks of cryptosporidiosis associated with drinking water supplies have been a major concern, and toxoplasmosis remains a serious infection for the fetus when acquired during pregnancy.

More than 340 parasitic species infect more than 3 billion people worldwide with varying morbidity and mortality. Examples of parasites include, but are not limited to Trypanosoma, Leishmania, Toxoplasma, Eimeria, Neospora, Cyclospora and Cryptosporidia. Acquisition of infection, clinical severity, and outcome of a parasitic disease depend on innate and acquired host immunity as well as the parasite's own immune response against the host when infection is established. Treatments are usually species specific, sometimes parasite stage specific, often expensive, and many parasites have become resistant to available drugs. Moreover, while treatments are available for some parasites, many anti-parasitic drugs have the potential for gastrointestinal, hepatic, renal, and hematologic toxicity and may interact with the metabolism of immunosuppressive agents.

It is therefore an object of the present invention to provide methods and compositions for treatment or prevention of parasitic diseases.

It is a further object of the present invention to provide methods and compositions for relatively inexpensive treatment of parasitic diseases.

### SUMMARY OF THE INVENTION

A method for using thiazine dyes, especially methylene blue, alone or in combination with low levels of light, to selectively inactivate or inhibit parasitic diseases is described. Examples of useful thiazine dyes are methylene blue, azure A, azure B, azure C, methylene green, new methylene blue, Taylor's blue, Toluidine Blue O, and thionine. The preferred dye at this time is methylene blue. Since methylene blue absorbs in the red wavelengths, i.e., approximately 670 nm, which penetrates tissue much better than other lower wavelengths, light penetrating the skin to the capillaries at the surface can be used to enhance the activity of the dye. The thiazine dye can be provided in combination with other known antibiotics anti-inflammatories, antifungals, anti-parasitics and antivirals.

### DETAILED DESCRIPTION OF THE INVENTION

1. Therapeutic Compositions

Thiazine Dyes

Examples of useful thiazine dyes includes, but are not limited to, methylene blue, methyl methylene blue, dimethyl methylene blue, azure A, azure B, azure C, methylene green, new methylene blue. Taylor's Blue, Toluidine Blue O, and thionine. Methylene blue is the preferred dye. These dyes are all commercially available from a number of different sources. Symmetrical 3,7-bis(dialkyl amino)phenothiazin-5ium derivatives which may be useful are described in Moura et al., Current Drug Targets, Vol. 4, 133-141 (2003).

### Methylene Blue and Its Derivatives

Methylene blue, 3,7-bis(dimethylamino)-phenothiazin-5-ium chloride, C₁₆H₁₈ClN₃S, is a dark green or blue thiazine dye which was first isolated in 1876. Methylene blue is a thiazine dye occurring as dark blue-green crystals which is soluble in water and sparingly soluble in alcohol, forming deep blue solutions. Methylene blue injectable has a pH of 3-4.5. The pKₐ is between 0 and −1.

Methylene blue has been approved for oral administration and has been reported to be effective as an antiseptic, disinfectant, and antidote for cyanide and nitrate poisoning. Methylene blue, injected i.v. at a dose of 1 mg/kg body weight, is effective in the treatment of methemoglobinemia, a clinical disorder where more than 1% of the hemoglobin in the blood has been oxidized to Fe³⁺. Drug Facts and Comparisons, page 1655 (J.B. Lippincott Co., St. Louis, Mo. 1989) reports that methylene blue is useful as a mild genitourinary antiseptic for cystitis and urethritis, in the treatment of idiopathic and drug-induced methemoglobemia and as an antidote for cyanide poisoning. Recommended dosages are 55 to 130 mg three times daily, administered orally. Oral absorption is 53% to 97%, averaging 74%, DiSanto and Wagner, J. Pharm. Sci. 61(7) 1086-1090 (1972). Pharmacopeia states that the recommended dose is 50 to 300 mg by mouth; 1 to 4 mg/kg body weight i.v. Side effects include blue urine, occasional nausea, anemia and fever. American Hospital Formulary Service “Drug Information 88” states that the recommended i.v. dosage for children is 1 to 2 mg/kg body weight, injected slowly over several minutes, which can be repeated after an hour. 55 mg tablets are available from Kenneth Manne. 65 mg tablets are available from Star Pharmaceuticals. Methylene Blue Injection (10 mg/ml) is available from American Reagant, Harvey, Kissimmee, Pasadena.

Narsapur anid Naylor reported in J. Affective Disorders 5, 155-161 (1953) that administration of methylene blue orally, at a dosage of 100 mg b.i.d. or t.i.d., or intravenously, 100 mg infused over 10 min, may be effective in treating some types of mental disorders in humans, indicating that the dye may cross the blood-brain barrier and therefore have particular applicability in the treatment of viral infections of the brain and central nervous system. Methylene blue was administered for periods of one week to 19 months to adult humans, with minimal side effects.

The American Hospital Formulary Service “Drug Information 88” reports that methylene blue is absorbed well from the GI tract, with about 75% excreted in urine and via the bile, mostly as stabilized colorless leukomethylene blue. As reported by G. E. Burrows in J. Vet. Pharmacol. Therap. 7, 225-231 (1984), the overall elimination rate constant of methylene blue, in sheep, is 0.0076±0.0016 min⁻¹, with minimal methemoglobin production at doses as high as 50 mg/kg and no hematologic changes seen up to four weeks after a total close of 30 mg/kg methylene blue. The 24 h LD₅₀ for intravenous methylene blue administered as a 3% solution was 42.3 mg/kg with 95% confidence interval limits of 37.3 to 47.9 mg/kg, demonstrating that methylene blue can be safely administered at a dosage of up to at least 15 mg/kj. As reported by Ziv and Heavner in J. Vet. Pharmacol. Therap. 7, 55-59 (1984), methylene blue crosses the blood-milk barrier easily.

U.S. Pat. No. 6,346,529 to Floyd, et al., describes the use of methylene blue and other thiazine dyes to inactivate HIV. It also demonstrates that the effect of the dye on different types of viruses is unpredictable, and that one cannot use results with one virus to predict efficacy with another. See Table 4, comparing efficacy against HIV with a lack of efficacy against Herpes Simplex Virus type 1 and type 2.

In contrast, U.S. Pat. No. 5,545,516 to Wagner, describes the inactivation of extracellular enveloped viruses in blood and blood components by phenthiazin-5-ium plus light. The described process inactivates pathogenic contaminants in whole blood, plasma, cellular blood component, by adding a phenthiazin-5-ium dye(s) thereto and irradiating the dye-containing composition with light of wavelengths from 560 to 800 nm or red light, such that they are suitable for transfusion. Obviously the conditions for treating blood products in a laboratory, and the availability of a radiant light source are quite different from the conditions required to treat a patient with a parasitic disease.

The compounds described herein have the chemical formula shown below: 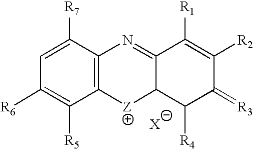
wherein R₁, R₂, R₄, R₅, and R₇ are independently selected from the group consisting of hydrogen, linear, branched or cyclic alkyl, aryl, substituted aryl, alkoxy, thioalkoxy, alkylamino, nitro, amino and halogen; R₃ and R₆ are independently selected from the group consisting of —O, —NH₂, —NHR₈, and —NR₉R₁₀ wherein R₈-R₁₀ is a linear, branched or cyclic hydrocarbon or R₉ and R₁₀ together with the nitrogen atom to which they are attached form an optionally substituted 5-, 6-, or 7-membered ring; wherein X⁻ is a counterion and wherein Z is either S or O.

Methylene blue, 3,7-Bis(dimethylamino)-phenothiazin-5-ium chloride, C₁₆H₁₈ClN₃S, is a dark green or blue thiazine dye. Derivatives of methylene blue in which the methyl groups of methylene blue have been replaced with ethyl, n-propyl, n-butyl, n-pentyl, and n-hexyl groups are described in Mellish et al., Photochemistry and Photobiology, Vol. 75, No. 4, pp. 392-397 (2002). Finally, phenoxazine dyes, in which the sulfur atom of the thiazine ring is replaced by an oxygen atom, may also be used. Examples of phenoxazine dyes include Nile Blue and its derivatives.

Methylene blue and its derivatives typically exist as the chloride or bromide salts; however, other anions can be used to stabilize the positive charge on the molecule. Suitable anions include inorganic anions such sulfate, sulfamte, phosphate, nitrate, and nitrite; and organic anions such as acetate, propionate, succinate, glycolate, stearate, lactate, malate, tartarate, citrate, ascorbate, pamoate, maleate, hydroxymaleate, phenylacetate, glutamate, benzoate, salicylate, sulfanilate, 2-acetoxybenzoate, fumarate, tolunesulfonate, napthalenesulfonate, methanesulfonate, ethane disulfonate, oxalate, and isethionate salts.

The activity of the dye can be enhanced further by irradiation with light or by derivatization with compounds such as antisense mRNA.

Formulations

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, and coating compositions.

“Carrier” also includes all components of the coating composition which may include plasticizers, pigments, colorants, stabilizing agents, and glidants. Delayed release dosage formulations may be prepared as described in references such as “Pharmaceutical dosage form tablets”, eds. Liberman et al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6^{th} Edition, Ansel et. al., (Media, Pa.: Williams and Wilkins, 1995) which provides information on carriers, materials, equipment and process for preparing tablets and capsules and delayed release dosage forms of tablets, capsules, and granules.

Examples of suitable coating materials include, but are not limited to, cellulose polymers such as cellulose acetate pthalate, hydroxypropyl cellulose, hydroxypropyl methylcellulose, hydroxypropyl methylcellulose pthalate and hydroxypropyl methylcellulose acetate succinate; polyvinyl acetate pthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

The amount of active agent released in each dose will be a therapeutically effective amount.

### Extended Release Dosage Forms

The extended release formulations are generally prepared as diffusion or osmotic systems, for example, as described in “Remington—The science and practice of pharmacy” (20the ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium, acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl pthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with suitable complexing agent.

The delayed release dosage units may be coated with the delayed release polymer coating using conventional techniques, e.g., using a conventional coating pan, an airless spray technique, fluidized bed coating equipment (with or without a Wurster insert), or the like. For detailed information concerning materials, equipment and processes for preparing tablets and delayed release dosage forms, see Pharmaceutical Dosage Form: Tablets, eds. Lieberman et al. (New York: Marcel Dekker, Inc., 1989), and Ansel et al., Pharmaceutical Dosage Forms and Drug Delivery Systems, 6 sup th Ed. (Media, Pa.: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

Alternatively, the dye can be continuously delivered to a patient over an extended period of time using a controlled release polymeric implant. Polymeric implants are generally manufactured from polymers which degrade in vivo over a known period of time. Examples of useful polymers include polyanhydrides, polylactic acid, polyorthoester, and ethylene vinyl acetate. The devices are also commercially available. Alza Corporation, Palo Alta, Calif., and Nova Pharmaceuticals, Baltimore, Md., both manufacture and distribute biodegradable controlled release polymeric devices.

The thiazine dyes can also be delivered using techniques known to those skilled in the art of drug delivery to target specific cell types or to enhance the activity of the dye. For example, a procedure utilizing injection of photoactive drugs for cancer treatment is described by Edelson, et al., in New England J. Med. 316, 297-303 (1987). Thiazine dye can be specifically delivered to macrophages, a site of high hepatitis virus concentration in hepatitis virus patients, using techniques such as liposome delivery. Liposomes are generally described by Gregoriadis, Drug Carriers in Biology and Medicine Ch. 14, 287-341 (Academic Press, NY, 1979). Methods for making light sensitive liposomes are described by Pidgeon, et al., in Photochem. Photobiol. 37, 491-494 (1983). Liposome compositions are commercially available from companies such as the Liposome Company, Inc., Princeton, N.J. Release of compounds from liposomes ingested by macrophages is described by Storm, et al., in Biochem. Biophys. Acta 965, 136-145 (1988).

II. Methods of Treatment

A. Parasitic Diseases that may be treatable

The thiazine dyes are administered to a patient in need of treatment or prophylaxis. The thiazine dyes can be administered to animals or humans. Preferably the thiazine dyes are administered for treatment of parasites.

Suitable diseases for treatment include parasites of the Trypanosoma, Leishmania, Toxoplasma, Eimeria, Neospora, Cyclospora and Cryptosporidia families. Parasites that cause Microsporidial infections, Malaria, visceral leishmaniasis often known as kalaazar, African sleeping sickness, toxoplasmosis, giardiasis and Chagas' disease are also suitable for treatment with thiazine dyes. Other Suitable parasites for treatment include, but are not limited to, Plasmodium vivax, Plasmodium falciparum, Plasmodium malariae, Plasmodium ovale, Trypanosoma protoza, Entamoeba histolytica, Trichomonas vaginalis, Giardia lamblia, Trypanosoma brucei gambiense, Trypanosoma brucei rhodesiense, Trypanosoma cruzi, Leishmania major, Leishmania tropica, Leishmania aethiopica, Leishmania infantum, Leishmania braziliensis, Leishmania mexicana, Leishmania amazonensis, Leishmania donovani-Leishmania infantum complex, Cryptosporidium parvum, Toxoplasma gondii, Encephalitozoon species, Nosema species and Septata intestinalis. Other parasites not listed here are known to one of ordinary skill in the art and can also be treated with thiazine dyes. As used herein “treatable” refers to the efficacy of the drugs in preventing or limiting infection, reproduction, or disease caused by the parasite. Treatable does not mean that the disease must be completely cured, since in some cases it may be sufficient to minimize symptoms or spread of the organisms while the host's immune system attacks the parasite.

Malaria, sleeping sickness and chagas disease are infectious diseases caused by any of various protozoa such as Plasmodium vivax, Plasmodium falciparum, Plasmodium malariae, Plasmodium ovale and trypanosoma protoza. The four principal species of Plasmodium that cause malaria are P. falciparum, P. vivax, P. ovale and P. malariae. Malarial infecting agents are parasitic in red blood corpuscles and are transmitted to birds and mammals by the bite of an infected Amopheles mosquito. Physiological consequences of malarial infection include fever, chills, anaemia, liver enlargement, encephalitis renal damage and death. Therefore, malaria parasite is one of the most important of human pathogens. The trypanosoma protozoa are also parasitic in the blood stream of mammals and may be transmitted by the bite of a tsetse fly.

Treatment of P. falciparum malaria in humans with chloroquine compound has been generally successful, but in recent years serious problems have arisen, see Center for Disease Control: Chemoprophylaxis of Malaria Morbidity Mortality Weekly Rep., 27 (Suppl.) 81-90 (1978). One problem is that the number of chloroquine resistant species is increasing in areas with already high incidence of the variants namely, Southeast Asia, Indonesia, Panama, and parts of the Indian subcontinent. See World Health Organization, W.H.O. Chronicles, 32, 9-17, (1978). In addition, chloroquine-resistant species have recently been reported for the first time in East Africa, See Fogh, S. et al., Trans. Royal Soc. Trop. Med. and Hygiene, 73, 228-229 (1979). The most commonly used alternative treatments, namely, quinine, primethamine and sulfadiazine, have shortcomings including adverse side-effects (some of them serious), as well as cost and availability. In addition, the use of primaquine for the eradication of the exoerythocytic forms in Ovale and Virax malaria, is not recommended in glucose-6-phosphate dehydrogenase deficiency, a condition that has a high incidence among Blacks and some Caucasian ethnic groups, see Clyde, D. F., Bull. W.H.O, 50, 243-249 (1974). Recently, Schirmer, et al., Redox Rep. 2003; 8(5):272-5, reported that Methylene blue has intrinsic antimalarial activity and it can act as a chloroquine sensitizer. In addition, methylene blue must be considered for preventing methemoglobinemia, a serious complication of malarial anemia. As an anti-parasitic agent, methylene blue is pleiotropic: it interferes with hemoglobin and heme metabolism in digestive organelles, and it is a selective inhibitor of Plasmodium falciparum glutathione reductase. The latter effect results in glutathione depletion which sensitizes the parasite for chloroquine action.

Entamoeba histolytica is the cause of amoebic dysentery producing severe infection of the intestines that can spread to the liver.

Trichomonas vaginalis is a common sexually transmitted organism causing trichomoniasis infection of the vagina and urethra. Giardia lamblia causes giardiasis producing symptoms of diarrhea and other intestinal disturbances. Infection arises from the ingestion of cysts, usually through contaminated water.

Trypanosoma brucei gambiense and T. brucei rhodesiense cause trypanosomiasis, more commonly known as African sleeping sickness. The disease is an arthropod (insect)-borne infections and is spread by the bite of the tsetse fly in which part of the trypanosome life cycle is completed. The eventual invasion of the central nervous system by the trypanosomes gives rise to a comatose state from which the common name for the disease is derived.

Trypanosoma cruzi causes Chagas' disease (American trypanosomiasis). The intermediate hosts in this case are triatomid bugs that feed off the blood of man. Infection results from the inoculation of an infected bug's faeces into the bite wound. Individuals who survive the acute stage of the disease are frequently left with chronic and progressive neuronal and smooth muscle lesions in the heart and gastrointestinal tract. T. cruzi has an extensive reservoir in wild and domestic mammals and therefore Chagas' disease is zoonotic (human infections that can be caught from animals).

Leishmania species cause leishmaniasis. The disease is spread by the bite of sandflies. In man, the promastigotes from the bite of the sandfly become ingested by macrophages and multiply within them as amastigotes. Cutaneous leishmaniasis occurs if the region of infection remains localized to the dermis as an open sore. In the Old World (Southern Europe, the Middle East, India, former USSR and parts of Africa) L. major, L. tropica, L. aethiopica and certain subtypes of L. infantum are responsible. In the New World (Mexico southwards and through South America) species responsible include L. brazilensis, L. mexicana and L. amazonensis. If the organism spreads, then mutocutaneous leishmaniasis can occur in which the nose, mouth and palate becomes destroyed. Infection with members of the L. donovani-L. infantum complex produce the systematic disease of visceral leishmaniasis often known as kalaazar that occurs with a global distribution seen in Old and New World leishmaniasis. The parasites multiply within the macrophages of the liver, spleen, bone marrow and other organs. Untreated, the disease is usually fatal. As with trypanosomiasis, leishmaniasis is zoonotic as many mammals harbor the parasite.

Cryptosporidium parvum causes diarrhea disease mainly in infants and small children. It is normally self-limiting but in the immunocomprised host the disease can be severe. C. parvum is usually passed to man in water containing oocysts of the organism.

Toxoplasma gondii causes the multi-organ infection of toxoplasmosis. The domestic cat is the definitive host for T. gondii from which man and other mammals can become infected. Infection commonly arises from the consumption of under cooked meat and in the healthy adult is usually asymptomatic. The most devastating form of toxoplasmosis is seen in congenital infection when a pregnant mother passes the organism to the fetus. This can result in severe abnormalities at birth.

Microsporidial infections have only recently been highlighted by the frequent recognition of these obligate intracellular parasites in material from patients with HIV infections and AIDS. Examples include: Encephalitozoon species, Nosema species and Septata intestinalis. Multi-organ infections occur and S. intestinalis is found in about 2% of all AIDS patients with chronic diarrhea.

B. Treatment Regimes

The drug is preferably administered orally, although it can also be administered by injection. The preferred dosage range for methylene blue is 30 to 180 mg twice a day, more preferably between 60 and 130 mg twice a day, or a dosage which yields blood levels between 0.2 and 2000 and more preferably between 2 and 200 microM methylene blue, administered orally in an immediate release formulation. The appropriate in vivo dosage can be determined by extrapolation from in vitro levels, assuming the usual blood volume for adult humans is approximately 10, and taking into account the 74% oral absorption and 75% excretion of that absorbed over a period of time, and assuming the lower therapeutic index in darkness than in light.

The method described herein does not require administration of exogenous light, although the results may be enhanced by exposure to light in addition to that normally transmitted through the skin. Exposure to light can occur with exposure to sun light, a tanning light, or even incandescent light.

### Combination Therapy

The thiazine dye can be provided in combination with other known antibiotics, anti-inflammatories, antifungals, anti-parasitics and antivirals to provide a combination therapy. Combination therapy is intended to include any chemically compatible combination of thiazine dye with other compounds, as long as the combination does not eliminate the activity of the thiazine dye.

For example, the thiazine dye can be used in combination with one or more other therapeutic agents, such as anti-inflammatory, anti-viral, anti-fungal, amoebicidal, trichomonocidal, analgesic, anti-neoplastic, anti-hypertensives, anti-microbial and/or steroid drugs, to treat antiviral infections. Suitable antibiotics include, but are not limited to, beta-lactam antibiotics, chloramphenicol, rifampin, clarithromycin, adriamycin, erythropoietin, neomycin, gramicidin, bacitracin, sulfonamides, and nalidixic acid. Suitable anti-inflammatory agents include, but are not limited to, cortisone, hydrocortisone, betamethasone, dexamethasone, fluocortolone, prednisolone, triamcinolone, indomethacin, sulindac. Suitable anti-fungals include, but are not limited to, voriconazole (VFEND®), azoles, imidazoles, polyenes, posaconazole, fluconazole, itraconazole, amphotericin B, 5-fluorocytosine, miconazole, and ketoconazole. Suitable antivirals include, but are not limited to, interferon, cyclovir, famciclovir or valacyclovir, alpha-interferon, ribavirin, and interferon or combinations of ribavirin and interferon or beta globulin.

Suitable anti-parasitic drugs include, but are not limited to, chloroquine, pyrimethamine-sulfasoxine, mefloquine, atovaquone, quinine primaquine, mebendazole, metranidazole, trimethoprim-sufamethoxazole, iodoquinol, suramine, pentamidine, melarsoprol, oxamniquine, praziqantel, nitazoxanide, pyrantel pamoate, albendazole, thiabendazole, pentavalent antimonilas pentostam, glucantine and diloxanide. In a preferred embodiment, methylene blue is used in combination with anti-parasitic drugs. Chloroquine doses for treatment of malaria usually involves 1 g of chloroquine to start, 500 mg six to eight hours after the first dose, and 500 mg once a day on the second and third days of treatment. Doses for melarsoprol used to treat African Sleeping Sickness are based on body weight and are determined by a physician. Mefloquine is normally administered once a week for one to three weeks prior to and four weeks after exposure to malaria in 1250 mg tablets as a single dose, or 750 mg as one dose, then a 500 mg dose 8 hours later.

Combination therapy can be sequential, meaning treatment with one agent first followed by treatment with a second agent, or it can be simultaneous, meaning treatment with both agents at the same time. If the combination therapy is sequential, administration of a second agent occurs within a reasonable time after administration of the first agent. If the combination therapy is simultaneous, both agents can be administered at the same time in the same dose or in separate doses. The exact regimen will depend on the severity of the disorder and the response to the treatment.

### Drug Resistance

Drug resistance is the result of microbes, such as parasites, changing in ways that reduce or eliminate the effectiveness of drugs, chemicals, or other agents to cure or prevent infections. Drug resistance can be considered as a natural response to the selective pressure of the drug. However, it is exacerbated by several factors, including abuse, underuse or misuse of the drug, poor patient compliance, and poor quality of available drugs.

According to the World Health Organization, resistance of Plasmodium falciparum to chloroquine, the cheapest and most used drug for treatment of malaria is spreading in almost all endemic countries. Resistance to the combination of sulfadoxine-pyrimethamine which was already present in South America and in South-East Asia is now emerging in East Africa. The problem of antimalarial drug resistance is aggravated by the existence of cross resistance among drugs belonging to the same chemical family.

Drug resistance can be classified into two categories, intrinsic or acquired. Drug resistance is considered intrinsic when parasites are intrinsically not sensitive to the drug (i.e. the parasite was never sensitive to the drug). Drug resistance is considered acquired when a normally sensitive parasite acquires resistance to the drug (i.e. the parasite is no longer sensitive to what is normally considered a toxic dose of the drug). Resistance to first line drugs has been observed in parasitic diseases such a trypanosomiasis, malaria and leishmania infections. Therefore, there is a need for additional drugs effective in treating parasitic infections. Methylene blue and its analogues as described herein can be used to treat drug resistant malaria, trypanosomiasis and other drug resistant parasitic infections in instances of intractability to normal therapy. Thiazine dye use can therefore be extended, but not limited to, the treatment of malaria resistant to chloroquine, trypanosomiasis resistant to melarsoprol and leishmania resistant to the pentavalent antimonilas pentostam and glucantine.

### Sensitizing Agent

Methylene blue and its analogues as described herein can also be used to sensitize non-drug resistant parasites to anti-parasitic drugs. A sensitizing agent is a drug that sensitizes an organism to its normal drug therapy. In other words, treatment of an organism with a sensitizing agent in combination with the normal drug therapy is more toxic to the organism than if the normal drug therapy was administered alone. For example, methylene blue can be used to sensitize malaria to chloroquine treatment.

Methylene blue and its analogues can also be administered as a sensitizing agent to parasites that are drug resistant. To overcoming drug resistance methylene blue or an analogue thereof can be administered to increase the sensitivity of the resistant parasitic strain to another anti-parasitic agent. When used a sensitizer, methylene blue can be administered prior to or simultaneously with the anti-parasitic agent.

We claim:

1. A method for treating parasitic disease in a patient comprising:
administering to the patient an effective amount of a thiazine dye in a pharmaceutically acceptable carrier to kill or inhibit infection of the parasite.

2. The method of claim 1 further comprising enhancing the anti-parasitic activity of the dye by exposure to non-ionizing radiation.

3. The method of claim 1 wherein the thiazine dye is selected from the group consisting of methylene blue, toluidine blue O, azure A, azure B, azure C, and combinations and derivatives thereof.

4. The method of claim 1 wherein the dye is methylene blue.

5. The method of claim 1 wherein the dye is administered orally.

6. The method of claim 1 wherein the dye is administered intravenously.

7. The method of claim 1 further comprising delivering the dye in a controlled release formulation.

8. The method of claim 1 further comprising providing the thiazine dye in combination with a compound selected from the group consisting of antibiotics, anti-inflammatories, anti-parasitics, antifungals, and antivirals.

9. The method of claim 8 wherein the anti-parasitic is selected from the group consisting of chloroquine, pyrimethamine-sulfasoxine, mefloquine, atovaquone, quinine primaquine, mebendazole, metranidazole, trimethoprim-sufamethoxazole, iodoquinol, suramine, pentamidine, melarsoprol, oxamniquine, praziqantel, nitazoxanide, pyrantel pamoate, albendazole, thiabendazole, pentavalent antimonilas pentostam, glucantine and diloxanide.

10. The method of claim 1 wherein the parasitic disease is selected from the group consisting of Microsporidial infection, Malaria, visceral leishmaniasis, African sleeping sickness, toxoplasmosis, giardasis and Chagas' disease.

11. A pharmaceutical composition for inhibiting or treating a parasitic disease comprising a therapeutically effective amount of a controlled release formulation comprising a thiazine dye in a pharmaceutically acceptable carrier, wherein the thiazine dye is selected from the group consisting of methylene blue, toluidine blue O, azure A, azure B, azure C, and combinations and derivatives thereof, to inhibit or prevent the parasitic disease.

12. The composition of claim 11 wherein the delivery system is a sustained or pulsed controlled release formulation.

13. The composition of claim 11 wherein the dye is in a composition for oral delivery.

14. The composition of claim 13 comprising a dosage equivalent to 130 mg twice a day orally.

15. The composition of claim 11 wherein the parasitic disease is selected from the group consisting of Microsporidial infection, Malaria, visceral leishmaniasis, African sleeping sickness, toxoplasmosis, giardiasis and Chagas' disease.
